(19) United States
(12) Patent Application Publication
(10) Pub. No.: US 2014/0132923 A1
(43) Pub. Date: May 15, 2014

(54) OPHTHALMIC IMAGING APPARATUS, CONTROL METHOD FOR OPHTHALMIC IMAGING APPARATUS, AND STORAGE MEDIUM

(71) Applicant: CANON KABUSHIKI KAISHA, Tokyo (JP)

(72) Inventors: Daisuke Kawase, Ichikawa-shi (JP); Osamu Sagano, Inagi-shi (JP)

(73) Assignee: CANON KABUSHIKI KAISHA, Tokyo (JP)

(21) Appl. No.: 14/063,103

(22) Filed: Oct. 25, 2013

(30) Foreign Application Priority Data
Nov. 9, 2012 (JP) 2012-247752

(51) Int. Cl. A61B 3/15

(52) U.S. Cl. CPC A61B 3/152
USPC 351/208; 351/246

(57) ABSTRACT

An ophthalmic imaging apparatus including an observation light source configured to emit observation light for observation of an eye to be examined and an imaging light source configured to emit imaging light for imaging the eye, the apparatus comprises a photometry unit configured to acquire a photometric value of an observation image of the eye illuminated by the observation light source; an alignment processing unit configured to perform alignment processing to match the eye with a target position; an imaging light amount adjusting unit configured to adjust a light amount of the imaging light source by using the photometric value before completion of the alignment processing; and an imaging processing unit configured to image the eye by using a light amount adjusted by the imaging light amount adjusting unit after completion of the alignment processing.

### BACKGROUND OF THE INVENTION

1. Field of the Invention

The present invention relates to an ophthalmic imaging apparatus, a control method for the ophthalmic imaging apparatus, and a storage medium.

2. Description of the Related Art

Some ophthalmic imaging apparatuses designed to image the fundus of an eye to be examined have an automatic imaging function of automatically performing imaging operation to reduce the operation burden on an operator when determining that the alignment state and in-focus state of the ophthalmic imaging apparatus are proper. Some other ophthalmic imaging apparatuses have an imaging light amount automatic adjustment function of automatically setting the imaging light amount of an imaging light source.

For example, Japanese Patent Laid-Open No. 2009-172154 discloses an ophthalmic imaging apparatus which automatically performs imaging operation when determining that check results on an alignment state and an in-focus state fall within proper ranges. Japanese Patent Laid-Open No. 2005-279154 discloses an ophthalmic imaging apparatus which causes an imaging light source to emit test light before actual imaging operation and decides an imaging light amount for actual imaging based on the reception result on reflected light. In addition, Japanese Patent Laid-Open No. 2012-050595 discloses an ophthalmic imaging apparatus which decides the light amount of an imaging light source based on the photometry result obtained by receiving the reflected light of observation light from the fundus of an eye to be examined.

As disclosed in Japanese Patent Laid-Open No. 2009-172154, when performing automatic imaging control based on check results on an alignment state and an in-focus state, an exposure result on a captured image does not sometimes match imaging conditions. In addition, as disclosed in Japanese Patent Laid-Open No. 2005-279154, the ophthalmic imaging apparatus configured to execute actual imaging upon causing the imaging light source to emit test light may impose burden on an object due to test light emission. As disclosed in Japanese Patent Laid-Open No. 2012-050595, the ophthalmic imaging apparatus configured to permit imaging operation upon acquisition of a stable photometry result does not sometimes permit imaging operation even when determining that an alignment state and an in-focus state are proper. This may lead to a deterioration in the operability of the ophthalmic imaging apparatus having the automatic imaging function.

The present invention provides an ophthalmic imaging technique which can obtain a captured image of an exposure state corresponding to imaging conditions without increasing the burden on an object while reducing the operation burden on the operator by using an automatic imaging function.

### SUMMARY OF THE INVENTION

According to one aspect of the present invention, there is provided an ophthalmic imaging apparatus including an observation light source configured to emit observation light for observation of an eye to be examined and an imaging light source configured to emit imaging light for imaging the eye, the apparatus comprising: a photometry unit configured to acquire a photometric value of an observation image of the eye illuminated by the observation light source; an alignment processing unit configured to perform alignment processing to match the eye with a target position; an imaging light amount adjusting unit configured to adjust a light amount of the imaging light source by using the photometric value before completion of the alignment processing; and an imaging processing unit configured to image the eye by using a light amount adjusted by the imaging light amount adjusting unit after completion of the alignment processing.

According to another aspect of the present invention, there is provided a control method for an ophthalmic imaging apparatus including an observation light source configured to emit observation light for observation of an eye to be examined and an imaging light source configured to emit imaging light for imaging the eye, the method comprising: a photometry step of acquiring a photometric value of an observation image of the eye illuminated by the observation light source; an alignment processing step of performing alignment processing to match the eye with a target position; an imaging light amount adjustment step of adjusting a light amount of the imaging light source by using the photometric value before completion of the alignment processing; and an imaging processing step of imaging the eye by using a light amount adjusted in the imaging light amount adjustment step after completion of the alignment processing.

According to the present invention, an ophthalmic imaging apparatus having a function of automatically adjusting an imaging light amount and an automatic imaging function can obtain a captured image of an exposure state corresponding to imaging conditions without increasing the burden on an object while reducing the operation burden on the operator by using the automatic imaging function.

Further features of the present invention will become apparent from the following description of exemplary embodiments (with reference to the attached drawings).

### BRIEF DESCRIPTION OF THE DRAWINGS

FIG. 1 is a view showing the arrangement of an ophthalmic imaging apparatus according to the first embodiment;

FIG. 2 is a view showing an observation state of the fundus of an eye to be examined in the ophthalmic imaging apparatus according to the first embodiment;

FIG. 3A is a flowchart for explaining a procedure for imaging processing in the ophthalmic imaging apparatus according to the first embodiment;

FIG. 3B is a block diagram for explaining the functional arrangement of an ophthalmic imaging apparatus according to the first embodiment;

FIG. 4 is a block diagram for explaining the functional arrangement of an ophthalmic imaging apparatus according to the second embodiment; and

FIG. 5 is a flowchart for explaining a procedure for imaging processing in the ophthalmic imaging apparatus according to the second embodiment.

### DESCRIPTION OF THE EMBODIMENTS

### First Embodiment

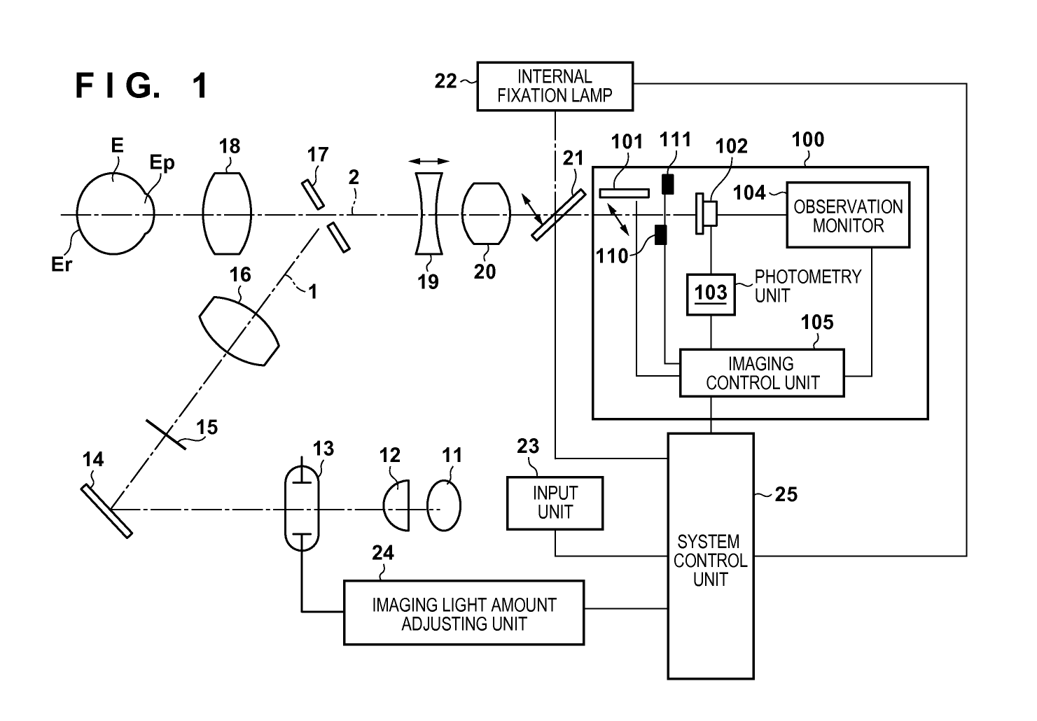

FIG. 1 is a view showing the arrangement of an ophthalmic imaging apparatus according to the first embodiment of the present invention. This embodiment will exemplify the arrangement of a non-mydriatic ophthalmic imaging apparatus as an ophthalmic imaging apparatus. A condenser lens 12, an imaging light source 13, a mirror 14, a stop 15 having a ring-like aperture, a relay lens 16, and a perforated mirror 17 are sequentially arranged on an optical path extending from an observation light source 11 to an objective lens 18 to constitute a fundus illumination optical system 1. A focusing lens 19, an imaging lens 20, and a flip-up mirror 21 are arranged on an optical path in the transmitting direction of the perforated mirror 17 to constitute a fundus imaging optical system 2 extending to an image sensor 102 placed in an imaging unit 100. An internal fixation lamp 22 including aligned/arranged light-emitting members such as LEDs for guiding the fixation of an eye E to be examined is placed in the reflecting direction of the flip-up mirror 21. In this case, the observation light source 11 is an LED light source which emits infrared light, and the flip-up mirror 21 is a mirror which transmits infrared light and reflects visible light.

Although not shown, an LED light source for alignment indices and the exit end of a light guide which guides a light flux from the LED light source are arranged in front of the perforated mirror 17 to constitute an alignment index projection system which projects alignment indices on the cornea surface of the eye E. Likewise, although not shown, the fundus illumination optical system 1 includes a focus index projection system which projects focus indices on a fundus Er of the eye E. An imaging light amount adjusting unit 24 which adjusts the light amount of the imaging light source 13 and an input unit 23 including an imaging start switch for capturing a still image of the fundus Er are connected to a system control unit 25 which controls the overall ophthalmic imaging apparatus.

The internal arrangement of the imaging unit 100 will be described next. The imaging unit 100 according to this embodiment is, for example, a removable single-lens reflex type digital camera. This is merely an example, and the scope of the present invention does not limit the arrangement of the imaging unit 100 to a single-lens reflex type digital camera. The imaging unit 100 incorporates an imaging control unit 105 which controls the imaging unit 100, the image sensor 102, a photometry unit 103 which calculates a photometric value corresponding to an output from the image sensor 102, and an observation monitor 104 such as an LCD. In the imaging unit 100, a flip-up mirror 101 and front and rear curtains 110 and 111 serving as shutter curtains are arranged in front of the image sensor 102. The imaging control unit 105 is connected to the flip-up mirror 101, the front curtain 110, the rear curtain 111, the photometry unit 103, and the observation monitor 104. The imaging control unit 105 controls the overall imaging unit 100, and is also connected to the system control unit 25 which controls the overall ophthalmic imaging apparatus.

The infrared light flux emitted by the observation light source 11 passes through the condenser lens 12 and the imaging light source 13 and is reflected by the mirror 14. The light reflected by the mirror 14 passes through the stop 15 and the relay lens 16 and is reflected by the periphery of the perforated mirror 17. This infrared light passes through the objective lens 18 and the pupil Ep of the eye E and illuminates the fundus Er. The infrared light reflected by the fundus Er illuminated with the infrared light passes through the pupil Ep of the eye E, the objective lens 18, and the hole of the perforated mirror 17 and is transmitted through the focusing lens 19, the imaging lens 20, and the flip-up mirror 21 to form an image on the image sensor 102. As described above, the infrared light emitted by the observation light source 11 is reflected by the fundus Er and is then formed into an image on the image sensor 102, which can be observed on the observation monitor 104. Retracting the flip-up mirror 101 from the fundus imaging optical system 2 and releasing the front curtain 110 and the rear curtain 111 at the time of observation can guide reflected light from the fundus Er to the image sensor 102. The photometry unit 103 performs photometry processing for an output from the image sensor 102. The imaging light amount adjusting unit 24 decides an imaging light amount corresponding to imaging conditions based on the photometry result obtained by the photometry unit 103.

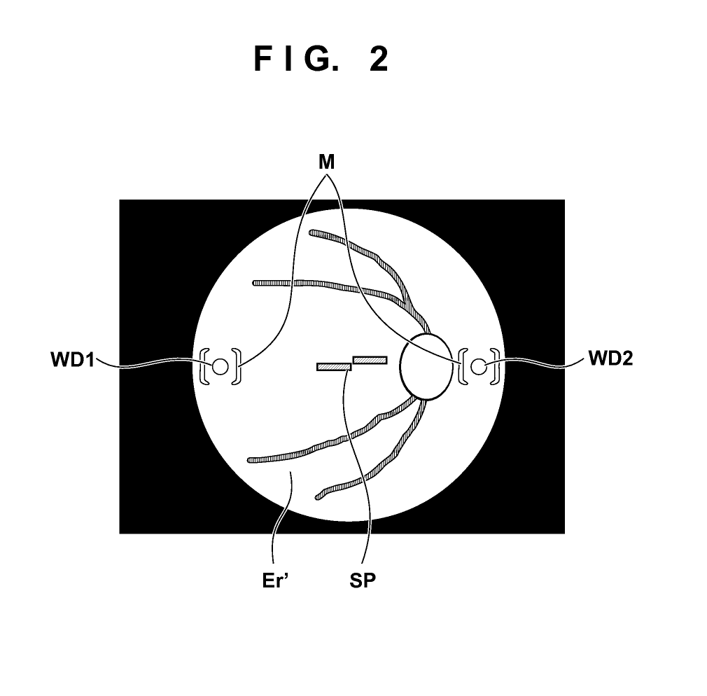

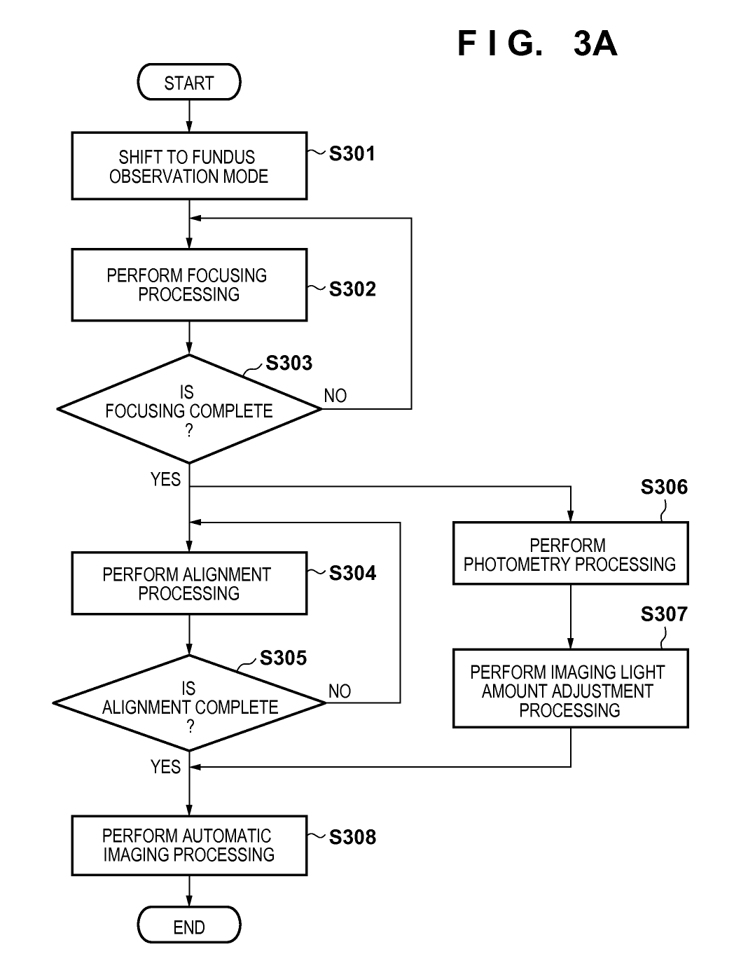

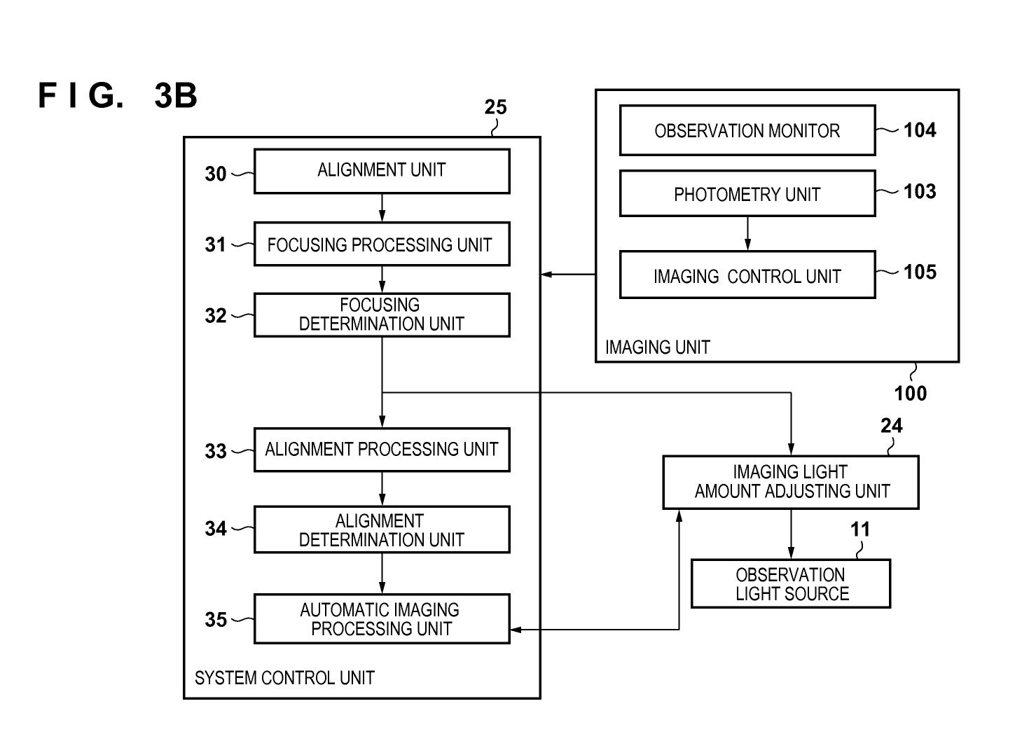

Light emerging from the alignment index projection system and the focus index projection system is reflected by the fundus Er and cornea surface of the eye to be examined and formed into images on the image sensor 102. It is possible to observe alignment indices WD1 and WD2 and focus indices SP on the observation monitor 104, together with an observation image Er′ of the fundus Er, as shown in, for example, FIG. 2. Referring to FIG. 2, reference symbol M denotes alignment positions. In alignment processing, the operator aligns the eye E with the ophthalmic imaging apparatus so as to set the alignment indices WD1 and WD2 at the alignment positions M. The operation of the operator and the operation of the ophthalmic imaging apparatus in this embodiment will be described next with reference to FIGS. 3A and 3B. FIG. 3A is a flowchart for explaining a procedure for imaging processing in the ophthalmic imaging apparatus according to the first embodiment. FIG. 3B is a block diagram for explaining the functional arrangement of the ophthalmic imaging apparatus.

First of all, an alignment unit 30 performs rough alignment between the ophthalmic imaging apparatus and the eye E during the observation of the anterior eye segment of the eye E (anterior eye segment observation mode). Upon completion of alignment, the system control unit 25 shifts the process in the ophthalmic imaging apparatus from the anterior eye segment observation state (anterior eye segment observation mode) to the fundus observation state (fundus observation mode) (step S301). Upon detecting the shift from the the anterior eye segment observation state (anterior eye segment observation mode) to the fundus observation state (fundus observation mode), a focusing processing unit 31 performs focusing processing (focus alignment) for the fundus Er of the eye E so as to align the focus indices SP (FIG. 2) in a line horizontally (step S302).

A focusing determination unit 32 determines whether a focusing processing result (focus adjustment result) falls within a proper range (threshold range) (step S303). If the focusing processing result does not fall within the threshold range (NO in step S303), the process returns to step 5302 to continue the focusing processing. If the focusing processing result falls within the threshold range (YES in step S303), the process advances to alignment processing (step S304) and photometry processing (step S306). The system control unit 25 can control the timing of processing by a photometry unit 103 with respect to an alignment processing unit 33. For example, the alignment processing unit 33 executes alignment processing (step S304). The photometry unit 103 executes photometry processing (step S306). The two processes are concurrently executed.

First of all, in the alignment processing in step 5304, the alignment processing unit 33 performs alignment between the eye E and the ophthalmic imaging apparatus to set the alignment indices WD1 and WD2 at the alignment positions M (target positions).

In step 5305, an alignment determination unit 34 determines whether the alignment result falls within a threshold range. If the alignment result does not fall within the threshold range (NO in step S305), the process returns to step 5304 to continue the alignment processing. If the alignment determination unit 34 determines that the alignment result falls within the threshold range (YES in step S305), the system control unit 25 shifts the process in the ophthalmic imaging apparatus to automatic imaging processing (step S308).

If the focusing processing result falls within the threshold range (YES in step S303), the system control unit 25 outputs a control signal to the imaging control unit 105 to start photometry processing in step S306. The imaging control unit 105 controls the operation of the photometry unit 103 in accordance with the control signal. The photometry unit 103 performs photometry processing based on the reflected light of observation light from the fundus Er based on an output from the image sensor 102.

In step S307, the imaging light amount adjusting unit 24 acquires a photometry result (photometric value) from the photometry unit 103 via the imaging control unit 105 and the system control unit 25. Before the completion of alignment processing after the focusing determination unit 32 determines that the focusing processing result falls within the threshold range, the imaging light amount adjusting unit 24 decides an imaging light amount for imaging processing based on the photometric value obtained by photometry by the photometry unit 103. In this case, the system control unit 25 can control the timing of processing by the imaging light amount adjusting unit 24 relative to processing by the alignment processing unit 33.

Although the arrangement configured to execute photometry processing in step S306 upon completion of focusing in step S303 has been described with reference to FIG. 3A, the scope of the present invention is not limited to this example. The embodiment of the present invention is configured to perform light amount adjustment by using a photometric value before the completion of alignment, and can also use, for example, a photometric value immediately before the completion of focusing instead of a photometric value after the completion of focusing in step S303. In this case, it is possible to perform imaging in a shorter period of time and obtain a captured image corresponding to imaging conditions without increasing the burden on an object.

Although the arrangement configured to execute imaging light amount adjustment processing in step S307 after the start of alignment processing in step S304 has been described with reference to FIG. 3A, the scope of the present invention is not limited to this example.

When performing photometry processing in step S306 by using a photometric value immediately before the completion of focusing, the imaging light amount adjusting unit 24 may acquire the photometry result obtained by the photometry unit 103 and concurrently perform alignment processing (step S304) and imaging light amount adjustment processing. The imaging light amount adjusting unit 24 can also execute imaging light amount adjustment processing at a timing before alignment processing (step S304).

If the focusing processing result falls within the threshold range, since the variation in light amount in the focus direction is small, alignment processing between the eye E and the ophthalmic imaging apparatus has a small influence on a photometry result. Using a photometry result on the reflected light of observation light from the fundus Er before imaging after focusing processing can acquire an effective photometry result without increasing the burden on the object.

In automatic imaging processing in step S308, an automatic imaging processing unit 35 controls the imaging light amount adjusting unit 24 to execute automatic imaging based on the decided imaging light amount. It is possible to perform automatic imaging based on a proper imaging light amount under the control of the automatic imaging processing unit 35 immediately after the alignment determination unit 34 determines that an alignment result falls within the threshold range. Although this embodiment has exemplified the non-mydriatic type ophthalmic imaging apparatus, the present invention is not limited to a non-mydriatic type ophthalmic imaging apparatus and can be applied to any type of ophthalmic imaging apparatus as long as it has a functional arrangement including an imaging light amount adjusting unit and a system control unit.

According to the present invention, the ophthalmic imaging apparatus having the function of automatically adjusting an imaging light amount and the automatic imaging function can obtain a captured image corresponding to imaging conditions without increasing the burden on an object while reducing the operation burden on the operator by using the automatic imaging function.

### Second Embodiment

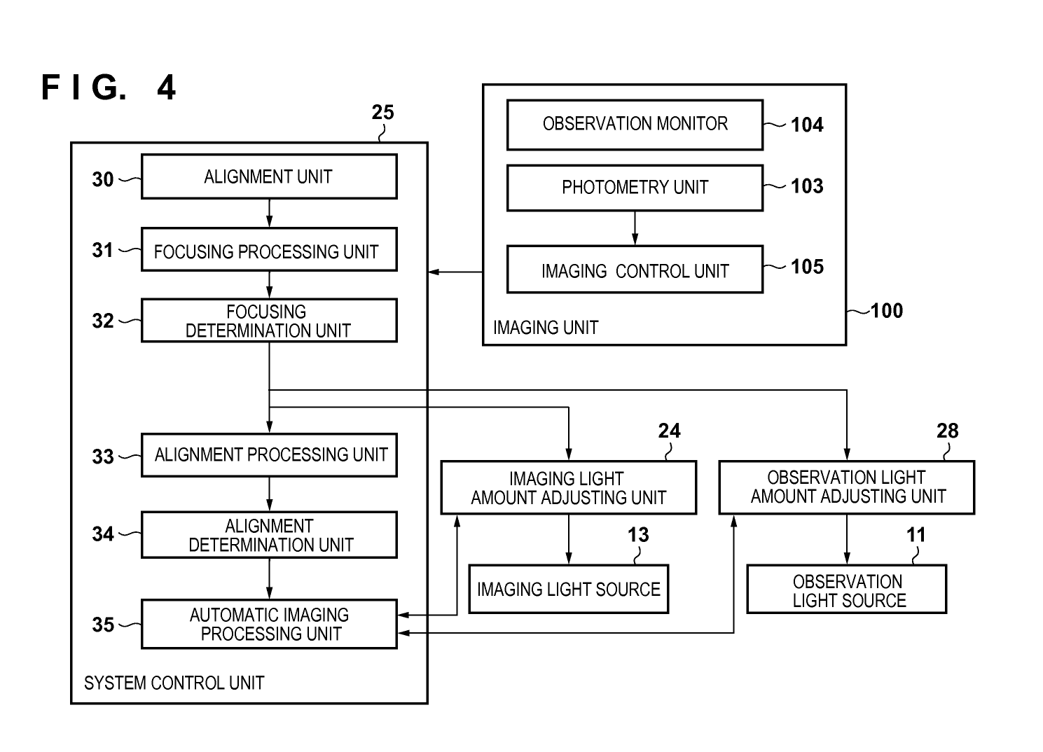

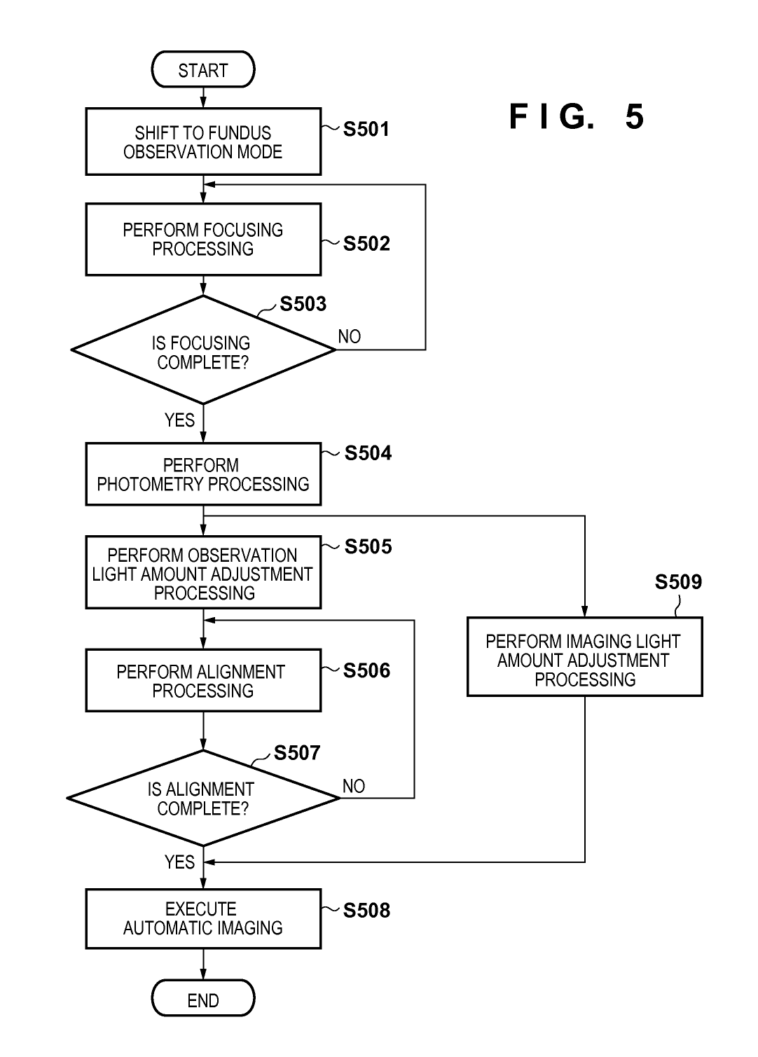

An ophthalmic imaging apparatus according to the second embodiment of the present invention will be described with reference to FIGS. 4 and 5. FIG. 4 is a block diagram for explaining the functional arrangement of the ophthalmic imaging apparatus according to the second embodiment. FIG. 5 is a flowchart for explaining a procedure for automatic imaging control in the ophthalmic imaging apparatus. As shown in FIG. 4, the ophthalmic imaging apparatus according to the second embodiment additionally includes an observation light amount adjusting unit 28 for adjusting the light amount of the observation light source 11. The same reference numerals as in the first embodiment denote the same constituent elements of the functional arrangement in FIG. 3B.

First of all, an alignment unit 30 performs rough alignment between the ophthalmic imaging apparatus and an eye E to be examined during the observation of the anterior eye segment of the eye E (anterior eye segment observation mode). Upon completion of alignment, a system control unit 25 shifts the process in the ophthalmic imaging apparatus from an anterior eye segment observation (anterior eye segment observation mode) to a fundus observation state (fundus observation mode) (step S501). Upon detecting the shift from the anterior eye segment observation state (anterior eye segment observation mode) to the fundus observation state (fundus observation mode), a focusing processing unit 31 performs focusing processing (focus adjustment) with respect to a fundus Er of the eye E so as to align focus indices SP in a line horizontally (step S502).

A focusing determination unit 32 determines whether the focusing processing result (focus adjustment result) falls within a threshold range (step S503). If the focusing processing result does not fall within the threshold range (NO in step S503), the process returns to step S502 to continue the focusing processing. If the focusing processing result falls within the threshold range (YES in step S503), the system control unit 25 outputs a control signal to a imaging control unit 105 to start photometry processing. The imaging control unit 105 then controls the operation of a photometry unit 103 in accordance with this control signal. In step S504, the photometry unit 103 performs photometry processing for the reflected light of observation light from the fundus Er based on an output from an image sensor 102.

The photometry processing result obtained in step S504 is used for observation light amount adjustment processing in step S505 and imaging light amount adjustment processing in step S509. The observation light amount adjusting unit 28 executes observation light amount adjustment processing (step S505). An imaging light amount adjusting unit 24 executes imaging light amount adjustment processing (step S509). These two types of processing are concurrently executed under the control of the system control unit 25.

First of all, in step S505, the observation light amount adjusting unit 28 acquires the photometry result (photometric value) obtained by the photometry unit 103 via the imaging control unit 105 and the system control unit 25. The observation light amount adjusting unit 28 calculates an observation light amount suitable for alignment processing, and generates an observation image suitable for the alignment processing. An observation monitor 104 displays an observation image of the fundus Er irradiated with adjusted observation light. After the observation light amount adjusting unit 28 terminates the observation light amount adjustment processing, the process advances to alignment processing in step S506.

In the alignment processing in step S506, an alignment processing unit 33 performs alignment between the eye E and the ophthalmic imaging apparatus so as to set alignment indices WD1 and WD2 at alignment positions M (target positions). In step S507, an alignment determination unit 34 determines whether the alignment result falls within a threshold range. If the alignment result does not fall within the threshold range (NO in step S507), the process returns to step S506 to continue the alignment processing. If the alignment determination unit 34 determines that the alignment result falls within the threshold range (YES in step S507), the process advances to automatic imaging processing in step S508.

In step S509, the imaging light amount adjusting unit 24 acquires the photometry result (photometric value) obtained by the photometry unit 103 via the imaging control unit 105 and the system control unit 25. After the focusing determination unit 32 determines that the focusing processing result falls within the threshold range before alignment processing is complete, the imaging light amount adjusting unit 24 decides an imaging light amount for imaging processing based on the photometric value obtained by photometry by the photometry unit 103. If the focusing processing result falls within the threshold range (YES in step S503), since the variation in light amount in the focus direction is small, alignment processing between the eye E and the ophthalmic imaging apparatus has a small influence on a photometry result. Using a photometry result on the reflected light of observation light from the fundus Er before imaging after focusing processing can acquire an effective photometry result without increasing the burden on the object. After the completion of the imaging light amount adjustment processing (step S509), the process advances to the automatic imaging processing in step S508.

In step S508, the automatic imaging processing unit 35 controls the imaging light amount adjusting unit 24 to execute automatic imaging based on the decided imaging light amount. It is possible to perform automatic imaging based on a proper imaging light amount adjusted by the imaging light amount adjusting unit 24 immediately after the alignment determination unit 34 determines that an alignment result falls within the threshold range.

This embodiment is configured to make the photometry unit 103 start photometry if the focusing determination unit 32 determines that a focusing processing result falls within the threshold range. However, the scope of the present invention is not limited to this arrangement. For example, the photometry unit 103 may always perform photometry, and the observation light amount adjusting unit 28 or the imaging light amount adjusting unit 24 may adjust an observation light amount or imaging light amount by using only a necessary photometric value. In addition, the apparatus is configured to start alignment processing after the observation light amount adjusting unit 28 completely adjusts an observation light amount for alignment processing. However, the scope of the present invention is not limited to this arrangement. For example, the apparatus may be configured to concurrently execute photometry processing, observation light amount adjustment processing, and alignment processing under the control of the system control unit 25 and properly reflect the observation light amount decided based on the photometry result obtained by the photometry unit 103 in alignment processing.

According to this embodiment, the ophthalmic imaging apparatus having the function of automatically adjusting an observation light amount and an imaging light amount and the automatic imaging function can provide an observation image corresponding to imaging conditions in each processing in the fundus observation state. In addition, it is possible to obtain a captured image in an exposure state corresponding to imaging conditions without increasing the burden on the object while reducing the operation burden on the operator by using the automatic imaging function.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-247752, filed Nov. 9, 2012, which is hereby incorporated by reference herein in its entirety.

What is claimed is:

1. An ophthalmic imaging apparatus including an observation light source configured to emit observation light for observation of an eye to be examined and an imaging light source configured to emit imaging light for imaging the eye, the apparatus comprising:
a photometry unit configured to acquire a photometric value of an observation image of the eye illuminated by the observation light source;
an alignment processing unit configured to perform alignment processing to match the eye with a target position;
an imaging light amount adjusting unit configured to adjust a light amount of the imaging light source by using the photometric value before completion of the alignment processing; and
an imaging processing unit configured to image the eye by using a light amount adjusted by said imaging light amount adjusting unit after completion of the alignment processing.

2. The apparatus according to claim 1, further comprising a focusing processing unit configured to adjust a focus with respect to the eye illuminated by the observation light source, wherein said photometry unit acquires a photometric value of an observation image of the eye illuminated by the observation light source after completion of adjustment of the focus.

3. The apparatus according to claim 1, further comprising an observation light amount adjusting unit configured to adjust a light amount of the observation light source based on the photometric value, wherein said alignment processing unit performs the alignment processing by using an observation image with a light amount adjusted by said observation light amount adjusting unit.

4. The apparatus according to claim 2, further comprising a focusing determination unit configured to determine whether an adjustment result on the focus falls within a threshold range,
wherein if the adjustment result on the focus falls within the threshold range, said photometry unit executes processing for acquiring the photometric value.

5. The apparatus according to claim 1, further comprising an alignment determination unit configured to determine whether a result on the alignment processing falls within the threshold range,
wherein if the result on the alignment processing falls within the threshold range, said imaging processing unit images the eye with the adjusted light amount of the imaging light source.

6. The apparatus according to claim 1, wherein said imaging light amount adjusting unit adjusts the light amount of the imaging light source by using the photometric value before completion of the alignment processing.

7. The apparatus according to claim 1, further comprising a control unit configured to perform control so as to execute processing by said photometry unit concurrently with processing by said alignment processing unit.

8. The apparatus according to claim 1, further comprising a control unit configured to perform control so as to execute processing by said imaging light amount adjusting unit concurrently with processing by said alignment processing unit.

9. A control method for an ophthalmic imaging apparatus including an observation light source configured to emit observation light for observation of an eye to be examined and an imaging light source configured to emit imaging light for imaging the eye, the method comprising:
a photometry step of acquiring a photometric value of an observation image of the eye illuminated by the observation light source;
an alignment processing step of performing alignment processing to match the eye with a target position;
an imaging light amount adjustment step of adjusting a light amount of the imaging light source by using the photometric value before completion of the alignment processing; and
an imaging processing step of imaging the eye by using a light amount adjusted in the imaging light amount adjustment step after completion of the alignment processing.

10. The method according to claim 9, further comprising a focusing processing step of adjusting a focus with respect to the eye illuminated by the observation light source,
wherein a photometric value of an observation image of the eye illuminated by the observation light source is acquired in the photometry step after completion of adjustment of the focus.

11. The method according to claim 9, further comprising an observation light amount adjustment step of adjusting a light amount of the observation light source based on the photometric value,
wherein the alignment processing is performed in the alignment processing step by using an observation image with a light amount adjusted in the observation light amount adjustment step.

12. The method according to claim 10, further comprising a focusing determination step of determining whether an adjustment result on the focus falls within a threshold range,
wherein if the adjustment result on the focus falls within the threshold range, processing for acquiring the photometric value is executed in the photometry step.

13. The method according to claim 9, further comprising an alignment determination step of determining whether a result on the alignment processing falls within the threshold range,
wherein if the result on the alignment processing falls within the threshold range, the eye is imaged with the adjusted light amount of the imaging light source in the imaging processing step.

14. The method according to claim 9, wherein the light amount of the imaging light source is adjusted in the imaging light amount adjustment step by using the photometric value before completion of the alignment processing.

15. The method according to claim 9, further comprising a control step of performing control so as to execute processing in the photometry step concurrently with processing in the alignment processing step.

16. The method according to claim 9, further comprising a control step of performing control so as to execute processing in the imaging light amount adjustment step concurrently with processing in the alignment processing step.

17. A non-transitory computer-readable storage medium storing a program for causing a computer to serve as each unit of an ophthalmic imaging apparatus according to claim 1.
